(19) United States
(12) Patent Application Publication
(10) Pub. No.: US 2003/0124080 A1
(43) Pub. Date: Jul. 3, 2003

(54) Personal care products

(76) Inventors: Antoine Kawam, Washington, DC (US); Richard E. Stafford, Arlington, VA (US)

Correspondence Address: FISH & RICHARDSON PC, 225 FRANKLIN ST, BOSTON, MA, 02110, US

(21) Appl. No.: 10/034,966

(22) Filed: Dec. 26, 2001

(51) Int. Cl. A61K 7/06

(52) U.S. Cl. 424/70.11

(57) ABSTRACT

The invention provides personal care products that include, within a pressurized can, a personal care formulation, a propellant, and a sorbant that has formed a gel with at least a portion of the propellant. The invention also provides methods of manufacturing personal care products.

### TECHNICAL FIELD

This invention relates to personal care products, such as shaving creams and antiperspirants.

### BACKGROUND

Personal care products that are sold in aerosol form, for example foam shaving creams, include propellants that are used to discharge the product from the container in which it is provided. The formulation, e.g., the shaving cream, is in an aqueous medium that generally does not blend with the liquified propellant.

In the case of foamed personal care products, e.g., shaving foams, the formulation is typically an aqueous soap solution and the propellant is typically a liquified hydrocarbon gas. The propellant is emulsified into the formulation, and therefore comes out of the container as a liquid. As the emulsion exits the container, the liquid propellant is converted to a gas, expanding the aqueous soap formulation to create a foam.

As the product is expelled from the container the volume of the headspace above the product in the container gradually increases. As a result, generally the propellant remaining in the container tends to vaporize into the increasing headspace, reducing the concentration of liquid propellant emulsified in the formulation. As a result, as the product is exhausted the density of the expanded foam (the foam density) will tend to increase and the foam will tend to gradually become more watery.

In the case of personal care products that exit the container in a non-foamed condition (referred to below as “non-foamed products”), for example shaving gels, the propellant generally should not be emulsified into the formulation, as this may result in the product exiting the container as a foam. In the case of shaving gels, the gel will typically contain a blowing agent that is emulsified into the formulation to cause the gel to foam when the blowing agent reaches skin temperature, resulting in foaming of the gel in the user's hand. Although these blowing agents may be similar in chemical composition to propellants, they are not used to expel the product from the container and thus are not considered “propellants” as that term is used herein, i.e., to refer to agents that are used to expel a formulation from a container.

Unless the propellant is separated from the gel formulation, the propellant will typically become emulsified in the formulation. Typically, the gel is provided in a two-compartment package, e.g., a bag within a can, in which an inner compartment contains the formulation and an outer compartment contains the propellant. The propellant compresses the inner compartment, causing the gel to be delivered from the container when a valve is actuated.

### SUMMARY

The present invention provides personal care products in which a propellant is at least partially sorbed and gelled by a sorbant. The product is provided in a pressurized container, from which it is discharged by a user. The sorbant does not exit the container with the product. Instead, the sorbant remains in the container to provide a reservoir of non-vaporized propellant.

In foamed products, the propellant is present in the container in three phases. A portion of the propellant is emulsified into the formulation as a liquid (the emulsified phase), so that the liquid propellant will expand upon exiting the container and foam the formulation. The remainder of the propellant is present in two additional phases: a gas phase, and a sorbed phase in which the propellant is sorbed onto the sorbant to form a gel.

The propellant gradually desorbs from the sorbant, as needed, to replace the propellant that is depleted as product is dispensed, as discussed above. This gradual desorption of propellant tends to maintain the equilibrium of the system within the container, and as a result the foam density tends to remain relatively constant as the product is exhausted. As a result, users can typically get more uses out of a can of the product, and will generally be more satisfied with the consistency of the foam that is delivered from a partially empty can.

In non-foamed products, the propellant is not emulsified as a liquid to any significant extent, but instead is present in only two phases: a gas phase, and a sorbed phase. The gaseous propellant acts as a plunger, forcing the formulation out of the container. As the gaseous propellant exits the can with the formulation, propellant is desorbed from the sorbant as a gas, maintaining the equilibrium within the container.

Thus, in the case of non-foamed products such as shaving gels, the use of a sorbant provides an alternative to dual-compartment packaging. Because the propellant is sorbed, it will not become emulsified with the formulation. The sorbant allows the propellant to be effectively separated from the product within a single-compartment container, simplifying the manufacturing process and reducing cost.

In some foamed products, the foam density of the product remains substantially constant (i.e., within ±0.05 g/cm³) until at least 70% of the contents of the can have been exhausted. Because the foam density remains relatively constant during much or all of the life of the product, users may tend to be more satisfied with the product, and may be able to use more of the contents of the container before discarding the product.

The propellant/sorbant system may also be used to deliver a low VOC aerosol personal care product, e.g., a low VOC aerosol antiperspirant spray, thereby reducing VOC emissions.

In one aspect, the invention features a personal care product including, within a pressurized container: (a) a personal care formulation; (b) a propellant; and (c) a sorbant that has formed a gel with at least a portion of the propellant.

Some implementations may include one or more of the following features. A first portion of the propellant is present in the container as a gas, and a second portion of the propellant is adsorbed onto the sorbant as a gel. The first and second portions may comprise substantially all of the propellant. Alternatively, a third portion of the propellant may be emulsified into the personal care formulation as a liquid. There is a gas/liquid phase equilibrium present in the container. The sorbant includes a polymer, which may be partially cross-linked, e.g., to an extent that will permit the polymer to swell upon adsorption of propellant, while remaining substantially insoluble in the propellant. The polymer is selected from the group consisting of silicones, polysiloxanes, polybutenes, polypropylenes, polyethylenes, and latex rubbers. The sorbant includes a foam or a fibrous material.

In another aspect, the invention features a personal care product including a single compartment container, and, within the container, a personal care gel formulation and a propellant, wherein substantially all of the propellant is either sorbed onto a sorbant or present in its gaseous phase.

In a further aspect, the invention features a method of manufacturing a personal care product including: (a) providing, within a container, a sorbant; (b) delivering, to the container, a personal care formulation; (c) sealing the container; and (d) pressurizing the container with a propellant.

The sorbant may be delivered to the container with, before, or after the delivery of the personal care formulation. Alternatively, the sorbant may be adhered to or coated on an inner surface of the container prior to delivery of the personal care formulation, or adhered to or coated on a dip tube inserted into the container.

Other features and advantages of the invention will be apparent from the description and drawings, and from the claims.

### BRIEF DESCRIPTION OF THE DRAWINGS

FIG. 1 is a graph showing shaving foam density measurements taken through the life of a can of a shaving foam product with a sorbant and a can of the same product without the sorbant.

FIG. 2 is a graph of measurements of the compression yield of foam samples taken through the life of a can of a shaving foam product with a sorbant and a can of the same product without the sorbant.

### DETAILED DESCRIPTION

Preferred personal care products include, a container and, within the container, an aqueous aerosol liquid or gel formulation, a propellant, and a sorbant onto which at least a portion of the propellant is adsorbed. As discussed above, the sorbant stays in the container throughout the life of the product and provides slow, controlled release of the propellant.

As discussed above, if it is desired that the formulation foam as it exits the container, e.g., if the product is a shaving foam, four phases are present in the container: (1) the liquid formulation, (2) a portion of the propellant present as a gas, (3) a portion of the propellant present as a liquid that is emulsified into the formulation to act as a blowing agent, and (4) a gel consisting of the sorbant and a portion of the propellant that is sorbed by the sorbant. The ratio of sorbed to liquid propellant will effect the properties of the dispensed product, e.g., the lather quality, and will effect the usable life of the product.

If foaming is not desired, e.g., if the product is a non-aerosol shaving gel, then phase (3) is not present, and instead all of the propellant is either in gaseous form or adsorbed onto the sorbant. Phase (3) can be eliminated by using an amount of propellant that is slightly less than the maximum capacity of the sorbant.

In both cases, there is a phase equilibrium present in the container.

Suitable formulations include but are not limited to solutions used in shaving foams; aqueous salt solutions, such as solutions containing an antiperspirant salt; and cream and gel personal care formulations. Suitable shaving compositions may include a water dispersible surface active agent dissolved or dispersed in water.

The water dispersible surface active agent may comprise a soap, a detergent, an anionic surfactant, a non-ionic surfactant, or a mixture of one or more of these. The soaps include, for example, the sodium, potassium and lower alkanolamine (preferably triethanolamine) salts of C10 to C20, preferably C12 to C18, fatty acids. Typical fatty acids include lauric, oleic, coconut oil, myristic, palmitic and stearic acid and mixtures thereof. The preferred fatty acids are palmitic and stearic. For purposes of the present invention, the water dispersible soaps are also intended to include the interrupted soaps such as the sodium, potassium and lower alkanolamine (preferably triethanolamine) salts of N-fatty acyl sarcosines wherein the fatty acyl moiety has 10 to 20, preferably 12 to 18, carbon atoms. Typical sarcosines include stearoyl sarcosine, myristoyl sarcosine, palmitoyl sarcosine, oleoyl sarcosine, lauroyl sarcosine, cocoyl sarcosine and mixtures thereof. The soaps (including the interrupted soaps) may be utilized in preneutralized form (i.e., as the sodium, potassium or alkanolamine salt) or in the free acid form followed by subsequent neutralization with sodium hydroxide, potassium hydroxide and/or alkanolamine (preferably triethanolamine). In any event, the composition must contain sufficient base to neutralize or partially neutralize the soap component and adjust the pH to the desired level.

The water dispersible surface active agent may also optionally include a non-ionic, amphoteric and/or anionic surfactant. Suitable non-ionic surfactants will typically have an HLB of 14 or more and include the polyoxyethylene ethers of fatty alcohols, acids and amides, particularly those having 10 to 20, preferably 12 to 18, carbon atoms in the fatty moiety and about 8 to 60, preferably 10 to 30, ethylene oxide units. These include, for example, PEG-150 Distearate, Oleth-20, Steareth-21, Ceteth-20, and Laureth-23. Other non-ionic surfactants include the polyoxyethylene ethers of alkyl substituted phenols, such as Nonoxynol-4 and Nonoxynol-20, fatty alkanolamides such as Lauramide DEA and Cocamide MEA, polyethoxylated sorbitan esters of fatty acids, such as Polysorbate-20, lauryl polyglucoside, sucrose laurate, and polyglycerol 8-oleate. Suitable amphoteric surfactants include, for example, the betaines and sultaines such as cocoamidopropyl betaine, coco dimethyl carboxymethyl betaine, coco sultaine and the like. Suitable anionic surfactants include, for example, the sodium, potassium, ammonium and substituted ammonium salts (such as the mono-, di- and triethanolamine salts) of C8-C22, preferably C12-C18, alkyl sulfates (e.g. sodium lauryl sulfate, ammonium lauryl sulfate), alkyl sulfonates (e.g. ammonium lauryl sulfonate), alkylbenzene sulfonates (e.g. ammonium xylene sulfonate), acyl isethionates (e.g. sodium cocoyl isethionate), acyl lactylates (e.g. sodium cocoyl lactylate) and alkyl ether sulfates (e.g. ammonium laureth sulfate). The surface active agent may typically include up to about 8% of non-ionic, amphoteric and/or anionic surfactants.

In addition to the surface active agent, the shaving composition may optionally include a variety of other well-known cosmetic ingredients generally known for use in shaving creams, foams and gels to improve the aesthetics and performance characteristics of the composition.

The shaving composition may contain about 1% to 10%, preferably about 1.5% to 7%, of a non-volatile paraffinic hydrocarbon fluid. The terms “non-volatile” and “fluid” mean that these materials are liquid at room temperature and have a boiling point above 200° C. Such hydrocarbon fluids include mineral oils and branched-chain aliphatic liquids. These fluids typically have from about 16 to about 48, preferably about 20 to about 40, carbon atoms and a viscosity of about 5 to about 100 cs., preferably about 10 to about 50 cs., at 40° C. The preferred non-volatile paraffinic hydrocarbon fluid is selected from mineral oil with a viscosity of about 10 to about 50 cs. at 40° C., hydrogenated polyisobutene with a molecular weight of about 320 to about 420, and mixtures thereof.

It may also be desirable to include a water-soluble gelling aid or thickening agent in the shaving composition to improve its consistency and stability, as well as to adjust its viscosity. These may include, for example, hydroxyalkyl cellulose polymers such as hydroxyethyl cellulose and hydroxypropyl cellulose (sold under the trademarks “Natrosol” and “Klucel” respectively), copolymers of acrylic acid and polyallyl sucrose (sold under the trademark “Carbopol”), carboxymethyl cellulose, and cellulose methyl ether (sold under the trademark “Methocel”). The gelling aid or thickening agent may be included in an amount of about 0.01% to 5%, preferably about 0.1% to 2%, by weight of the composition. The shaving composition may also include up to 8%, preferably about 2% to 6%, by weight of a fatty alcohol such as myristyl, lauryl and stearyl alcohol and octyl dodecanol. The term “fatty” is intended to include 10 to 20, preferably 12 to 18, carbon atoms.

Other useful additives which may be utilized in the composition include humectants such as glycerin, sorbitol, and propylene glycol, emollients including fatty esters such as isopropyl myristate, decyl oleate, 2-ethylhexyl palmitate, PEG-7 glyceryl cocoate, and glyceryl linoleate, propoxylated fatty ethers such as PPG-10 cetyl ether and PPG-11 stearyl ether, di- and triglycerides such as lecithin and caprylic/capric triglyceride, vegetable oils, and similar materials, skin freshening and soothing agents such as menthol, aloe, allantoin, lanolin, collagen and hyaluronic acid, fluorosurfactants, silicones (e.g. dimethicone, dimethiconol, dimethicone copolyol, stearyl dimethicone, cetyl dimethicone copolyol, phenyl dimethicone, cyclomethicone, etc.), vitamins (including vitamin precursors and derivatives) such as panthenol, vitamin E, tocopherol acetate, and vitamin A palmitate, colorants, fragrances, antioxidants and preservatives.

If the shaving composition is in the form of a self-foaming shave gel, it will include a blowing agent which may be any volatile hydrocarbon or halohydrocarbon with a sufficiently low boiling point that it will volatilize and foam the gel upon application to the skin, but not so low that it causes the gel to foam prematurely. The typical boiling point of such an agent generally falls within the range of −20° to 40° C. Preferred blowing agents are selected from saturated aliphatic hydrocarbons having 4 to 6 carbon atoms, such as n-pentane, isopentane, neopentane, n-butane, isobutane, and mixtures thereof. Most preferred is a mixture of isopentane and isobutane in a weight ratio (IP:IB) of about 1:1 to about 9:1, preferably about 2:1 to about 7:1. The blowing agent will normally be present in an amount comprising about 1% to about 6% of the composition, preferably about 2% to about 5%.

If the shaving composition is in the form an aerosol foam, it will include a propellant of sufficient volatility or pressure to propel the shaving composition from its container and cause it to foam. Typical propellants include liquifiable gas propellants such as volatile hydrocarbons, halohydrocarbons, and mixtures of hydrocarbons (typically with 3 to 6 carbon atoms). Generally, suitable propellants have a vapor pressure of 30 to 60 psig at about 20° C. A preferred propellant has the industry designation A-46 and is a mixture of n-butane, isobutane and propane with a vapor pressure of 46 psig at about 20° C. Another preferred propellant is isobutane (e.g., Aeron A-31 propellant, commercially available from Diversified CPC International (www.diversifiedcpc.com)). Other suitable propellants include propellant 152A and A-70.

In four-phase systems, the contents of the container, as sold, will generally include from about 2 to 80% propellant by weight, with the balance being the product formulation. Preferably, the contents include from about 3 to 20% propellant. For a shaving cream product, preferably the contents include from about 4 to 10% propellant.

In three-phase systems, the contents of the container, as sold, will generally include from about 2 to 20% propellant by weight, with the balance being the product formulation. Preferably, the contents include from about 5 to 10% propellant.

Suitable sorbants include polymeric open and closed cell foams and polymeric fibrous materials. Open cell foams are generally preferred due to their higher capillarity, and because open cells tend to speed sorption and desorption of the propellant.

Examples of suitable polymer foams include silicone and polysiloxane foams (e.g., BF-1000 foam, Rodgers Corp., Elk Grove Village, Ill.), polybutene foams, polypropylene foams (commercially available from Dupont), polyethylene foams (e.g., Minicel L200 foam, Voltek), and latex rubber foams. Suitable fibrous polymeric materials include polypropylene fibers.

The sorbant generally should have sufficient structural integrity so that it will not exit the container with the product to any significant extent. The sorbant may, for example, float loose in the container, be adhered to or coated on the inner side and/or bottom walls of the container, or be adhered to or coated on the outer surface of the dip tube that is disposed within the container and used to dispense the product from the container. For a typical shaving gel or foam container (can), the volume displaced by the sorbant within the container is generally about 0.5 to 2 cm³. If the sorbant floats loose, the foam should generally be cut into small pieces. The pieces should be sufficiently small so as to provide adequate surface area for sorption/desorption, but sufficiently large so that they will not interfere with dispensing, e.g., by clogging the dip tube or valve of the container.

Suitable sorbants generally will have a solubility parameter that is closely matched to that of the propellant. Preferably, the solubility parameters of the sorbant and the propellant differ by less than 2, more preferably by less than 0.5.

Sorption of the propellant by the sorbant is enhanced by capillary adsorption into the foam cells and/or along the fibers of the polymer. Generally, smaller cell size foams will exhibit greater capillary absorption. Sorbants that have solubility parameters that are significantly different from those of the propellant may nonetheless be suitable due to capillarity of the polymer material.

Because the sorbant should be swelled by the propellant to form a gel, rather than dissolved by the propellant, the sorbant should be cross-linked sufficiently so that it will be substantially insoluble in the propellant. A non-crosslinked polymer with a solubility parameter very close to that of the propellant would be dissolved by the propellant. On the other hand, the sorbant should not be cross-linked so much that it will not be able to adsorb the propellant. The cross-links may be made by covalent bonds, ionic coordination bonds, hydrogen bonds, or crystallites.

Suitable sorbants are described, e.g., in U.S. Pat. Nos. 3,813,041, 3,950,960 and 3,891,147, the disclosures of which are incorporated herein by reference.

Preferably, the ratio of propellant weight to sorbant weight is greater than 4:1, more preferably greater than 6:1, and most preferably greater than 8:1.

The product may be manufactured by any suitable method, including the methods described in the Examples below. In some methods, the polymer sorbant is placed inside the container, the product is added, the container is sealed, and the propellant is pressure loaded.

### EXAMPLES

In the following examples, the symbol δ_{g} indicates the solubility parameter of the sorbant, expressed in (Cal/cm³) 0.5.

### I. Four-Phase (Foaming) Systems

### Example 1

In this example, an anionic surfactant-based shaving cream was made, having the following formulation:

| Ingredient | Weight Percent |
|---|---|
| Water | 83.98 |
| Stearic Acid | 6.29 |
| Laureth-23 | 2.08 |
| Sodium lauryl sulfate | 0.78 |
| Triethanolamine (99%) | 3.21 |
| BHT | 0.02 |

This shaving cream was prepared as follows. The water was heated to 80-85 C., after which the stearic acid was added. Once the stearic acid had melted, the laureth-23 was added, melted, and mixed well. Next, triethanolamine was added and the resulting composition mixed well for about 30 minutes to form a soap. The resulting soap was cooled to about 65□C., after which sodium lauryl sulfate was added and the composition mixed well. Next, the BHT was added, followed by mixing.

One gram of an open-celled 12-lb/ft³⁻ polysiloxane foam, δ_{g}=7.3, (BF-1000), Rodgers Corp., Elk Grove Village, Ill.) was cut into small pieces. The polysiloxane foam was placed in a standard aerosol can, the formulation was added, the can was sealed, and the propellant was pressure loaded. After filling, the can was shaken for 5 minutes at room temperature. The dispensed product was a soap foam and hydrocarbon gas.

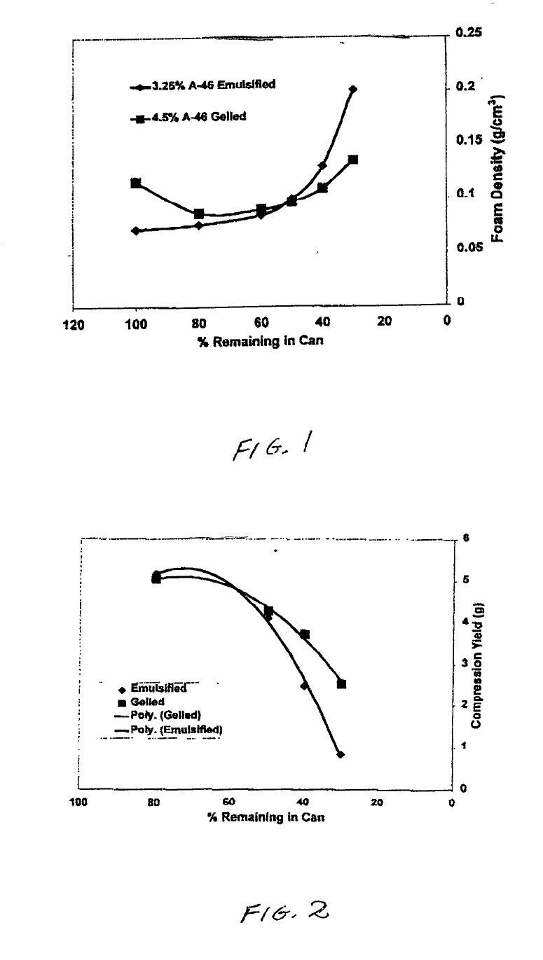

FIG. 1 is a graph showing shaving foam density measurements through life of a can of the product with the sorbant and a can of the same product without the sorbant. The density of the shaving foam was measured by standard methods. The cans were emptied 5 grams at a time with a 2-hour equilibration time between each actuation of the product. The can containing the sorbant produced a more consistent foam throughout the life of the can than the can that did not include a sorbant. In addition, the foam produced by the sorbant-containing product was creamier, richer, easier to spread, and more stable than the foam produced by the non-sorbant-containing product. FIG. 2 is a graph of measurements of the compression yield of foam samples produced through the life of the cans with and without sorbant.

### Example 2

Using the product described in Example 1, and an identical product without sorbant, a shave study was performed to see if measurable differences between the foams were perceived by male shavers. In this study the aesthetics of the gelled and emulsified propellant products were matched in the fullest cans (80% full) so the shavers found both products equally acceptable. Shaving split-face, male shavers were asked to rate the shaving creams with different amounts of product in the can. The panelists' overall perceptions of both shaving creams were generally favorable until the cans were 70% empty (30% full). At 30% full the panelists generally began to find the product without sorbant unacceptable.

These findings were supported by the results of an unrestricted home use test that investigated the perception of male shavers of foam products produced from partially empty cans. The subjects started the test with cans that were 37% full (this is the fullest level at which panelists in split-face test found the overall lather quality of the emulsified product neither acceptable nor unacceptable). Subjects were then asked to use and rate the product until the can was empty. For both products, about 8% of the formulation remained in the can when no more product could be expelled.

The average overall lather quality and average overall rating for the gelled propellant product were acceptable throughout the life of the sorbant-containing product while the non-sorbant-containing product rating dropped into the unacceptable range after the first shave. The ratings of shave attributes for the last shave shows that the men found the sorbant-containing product acceptable to the last dispensable portion of the formulation.

### Example 3

The formulation and manufacturing procedures of Example 1 were repeated, except the foam sorbant was a closed cell 12 lb/ft³ polysiloxane foam, δ_{g}=7.3, (ACME Corp) and was used at a level of 1 g foam to 10 g propellant. Gelling efficiency of this sorbant is 1 g sorbant per 10 g propellant.

### Example 4

The formulation and manufacturing procedures of Example 1 were repeated, except the sorbant was a non-woven polypropylene fiber, σ_{g}=8, manufactured by the 3M Corp., and was used at a level of 1 g foam to 10 g propellant. Gelling efficiency of this sorbant is 1 g sorbant per 10 g propellant.

### Example 5

The formulation and manufacturing procedures of Example 1 were repeated, except the sorbant was an open-celled latex rubber 8 lb/ft³ foam, σ_{g}=8.5, (Latex/SK, Rodgers Foam Corp.), and was used at the level of 1 g foam to 10 g propellant. Gelling efficiency of this sorbant is 1 g sorbant per 10 g propellant.

### Example 6

The formulation and manufacturing procedures of Example 1 were repeated, except the sorbant was a closed cell peroxide cross-linked polyethylene (88%) vinyl acetate (12%) 2 lb/ft³foam, 67_{g}=8 (commercial name Volara) manufactured by Voltek a division of Sekisui America Corp., and was used at a level of 1 g foam to 8 g propellant. Gelling efficiency of this sorbant is 1 g sorbant per 8 g propellant.

### Example 7

The formulation and manufacturing procedures of Example 1 were repeated, except the sorbant was a closed cell radiation cross-linked polyethylene 2 lb/ft³foam, δ_{g}=7.8, (commercial name Minicel L200) manufactured by Voltek a division Sekisui America Corp. and was used at a level of 1 g foam to 5 g propellant. Gelling efficiency of this sorbant is 1 g sorbant per 5 g propellant.

### Example 8

An aerosol spray shaving foam was prepared having the following formulation:

Aqueous concentrate

| Ingredient | Wt (%) |
|---|---|
| Water | 88.25 |
| Stearic Acid | 4.18 |
| Laureth-23 | 1.38 |
| Sodium Lauryl Sulfate | 0.52 |
| Triethanolamine (99%) | 2.16 |
| BHT | 0.01 |
| Fragrance | 0.50 |

Aerosol formulation

| Ingredient | | |
|---|---|---|
| Silicone foam BF-1000 | 0.13 | g/g A-31 |
| (Rodgers Corp.) | | |
| Aqueous Concentrate | 97 | wt % |
| Aeron A-31 Propellant | 3 | wt % |

The water was heated to 80-85□ C., after which stearic acid was added. Once the stearic acid had melted, the laureth-23 was added, melted, and mixed well. Next, triethanolamine was added and the resulting composition was mixed well for about 30 minutes to form a soap. The resulting soap was cooled to about 65□ C., after which sodium lauryl sulfate was added and the composition was mixed well. Next, the BHT was added, followed by mixing.

One gram of an open-celled silicone foam (BF-1000, Rodgers Corp., Elk Grove Village, Ill.) was cut into small pieces. The foam sorbant was placed in the can and then the aqueous concentrate was added. The can was a standard aerosol can provided with a valve-actuator system that included an upright-inverted valve (Summit UI-3, available from Summit Packaging Systems, Inc.) and a two-piece mechanical break-up actuator (No. 77902 with insert 70151-2402, also available from Summit). The specification for this valve is Stem 1×0.018, meaning that the valve stem has a single opening measuring 0.018 inch (0.046 cm). The can was sealed and then the propellant was pressure loaded. After filling, the can shaken for 5 minutes at room temperature. The dispensed product was a soap foam and hydrocarbon gas.

In this example enough foam sorbant was added to just gel all of the added propellant.

Adding more or less propellant results in denser or lighter foams, respectively.

### Example 9

A post-foaming shaving gel, i.e., a gel that foams in the user's hand, which can be dispensed from a standard aerosol can was made, using the following formulation:

Emulsion concentrate

| | |
|---|---|
| Palmitic acid | 5% |
| Stearic acid | 0.7% |
| Lauramide DEA | 2.8% |
| Lauryl Amine Oxide | 2.5% |
| Prisorine 2034 | .25% |
| Triethanolamine | 2.9% |
| Isopentane (blowing agent) | 2% |

Aerosol formulation

| Ingredient | | |
|---|---|---|
| Silicone foam BF-1000 | 1-2 | grams |
| Emulsion Concentrate | 97 | wt % |
| Aeron A-31 Propellant | 3 | wt % |
| (isobutene) | | |

The palmitic acid, stearic acid, lauramide DEA, lauryl amine oxide and Prisorine were dispersed in water and then heated to 80□ C. until melted. The triethanolamine was then added and mixed. The mixture was then cooled to 20□ C. and the isopentane was slowly stirred into the mixture. The aerosol formulation was filled two different ways using a standard aerosol can with a gel valve and gel actuator:

Fill method 1: 97 grams of the emulsion concentrate was poured into a standard aerosol can. Then 1 to 2 grams of the silicone foam, in the form of a pad, was added to the top of the formulation. The can was sealed and then 3 grams of isobutene (A-31) was pressure filled through the valve. The can was shaken about 60 seconds to move the A-31 to the top of formulation where it was gelled by the silicone pad. The dispensed product was a bead of gel that expanded into a foam in the user's hand, due to expansion of the isopentane and isobutene at skin temperature.

Fill method 2: One to two grams of silicone foam, in the form of a pad, was added to the can prior to charging the formulation to the can. The dip tube was threaded through the silicone pad, holding the pad at the top of the can. The can was sealed, evacuated, and then 3 grams of isobutene (A-31) was pressure filled through the valve. Then 97 g of the aerosol formulation was pressure filled through the valve. The dispensed product was a bead of gel that expanded into a foam in the user's hand.

### Example 10

A foaming product was made using the following formulation, which contained non-ionic surfactants:

Aqueous concentrate

| | Wt % |
|---|---|
| Polyoxyethylene (4) lauryl ether | 1.6 |
| Cetyl Stearyl alcohol | 1.2 |
| Water | 87.2 |

Aerosol formulation

| | | |
|---|---|---|
| Silicone foam BF-1000 | 0.12 | g/g of A-46 |
| (Rodgers Corp.) | | |
| Aqueous Concentrate | 95 | wt % |
| A-46 | 5 | wt % |

Preparation Method

The polyoxyethylene and the alcohol were added to water, heated to 80□ C., and melted. The dispersion was then removed from the heat and cooled while stirring. The cooled concentrate was poured into a standard aerosol can, followed by the silicone foam. The can was sealed and then the propellant was pressure filled.

### II. Three Phase Systems

### Example 12

In this example the sorbant was a large open-celled silicone foam prepared with the General Electric Silicones RTF7000 variable density silicone foam system, and the formulation was an aqueous anti-perspirant composition.

The silicone foam sorbant was prepared using the following materials and reaction conditions:

| Component | Parts by weight |
|---|---|
| D1-RTF7000 | 100 |
| SS4300C | 7.5 |
| Methanol | 5 |

The methanol was mixed into the D1-RTF7000 base at room temperature. The cross-linker SS4300C was then quickly mixed into the base. The mixture was then poured into a plastic container and cured at 40□ C. The foam core used for this example had 5 to 10 cells per inch and had a density of 10 lb/ft³.

Aqueous concentrate

| | Wt % |
|---|---|
| APACHE | 15.0 |
| (Aluminum Chlorohydrate AP salt) | |
| Ethyl alcohol | 20.0 |
| Water | 65.0 |

Aerosol formulation

| | | |
|---|---|---|
| Polysiloxane foam | 0.2 | g/g A-46 |
| (RTF7000) | | |
| Aqueous Concentrate | 95 | wt % |
| A-46 | 5 | wt % |

The large open cells of this foam sorbant allowed for quick equilibration of the gelled propellant with the gas phase. As a result, it was not necessary to cut the foam into small pieces. The cells were also large enough so that the aqueous concentrate could flow through the foam, and so that the foam exhibited little or no capillarity. A single circular plug of foam was cut to the appropriate weight. The foam plug was threaded onto the dip tube and then placed into a standard aerosol can. The valve in this case had no vapor tap. The can was fitted with a valve and actuator to produce a fine even spray. The can was sealed, evacuated and then the propellant was pressure filled, followed by the aqueous concentrate.

Other embodiments are within the scope of the following claims.

For example, any material that has compatible solubility can be gelled in the sorbant. For example, the sorbant can be used to deliver active materials into a formulation. A single sorbant material can be used to sorb both the propellant and any other material(s) to be sorbed, or the product can include two or more different sorbants. In the latter case, the sorbants can be selected to have different solubility parameters that are matched or similar to the solubility parameters of the propellant and other material(s).

Generally, if other materials are to be sorbed in addition to the propellant, the product contains from about 1 to 20% sorbant by weight, more preferably from 2 to 15%.

One material that may be added to shaving cream formulations and sorbed by the sorbant is polydimethylsiloxane. In this case, about 3 to 8% sorbant can be used, and about 3 to 8% of the polydimethylsiloxane. Addition of polydimethylsiloxane generally produces a creamy, dense foam that leaves a smooth skin feel.

What is claimed is:

1. A personal care product comprising, within a pressurized container:
a personal care formulation;
a propellant; and
a sorbant that has formed a gel with at least a portion of the propellant.

2. The personal care product of claim 1 wherein a first portion of the propellant is present in the container as a gas, and a second portion of the propellant is adsorbed onto the sorbant as a gel.

3. The personal care product of claim 2 wherein the first and second portions comprise substantially all of the propellant.

4. The personal care product of claim 2 wherein a third portion of the propellant is emulsified into the personal care formulation as a liquid.

5. The personal care product of claim 4 wherein there is a gas/liquid phase equilibrium present in the container.

6. The personal care product of claim 1 wherein the sorbant comprises a polymer.

7. The personal care product of claim 6 wherein the polymer is partially cross-linked.

8. The personal care product of claim 7 wherein the polymer is cross-linked to an extent that will permit it to swell upon adsorption of propellant, while remaining substantially insoluble in the propellant.

9. The personal care product of claim 6 wherein the polymer is selected from the group consisting of silicones, polysiloxanes, polybutenes, polypropylenes, polyethylenes, and latex rubbers.

10. The personal care product of claim 1 or 9 wherein the sorbant comprises a foam or a fibrous material.

11. The personal care product of claim 6 wherein the solubility parameter of the polymer is within 2 units of the solubility parameter of the propellant.

12. The personal care product of claim 1 or 4 wherein the personal care formulation comprises a soap solution.

13. The personal care product of claim 1 or 3 wherein the personal care formulation comprises a gel.

14. The personal care product of claim 1 wherein the personal care formulation comprises an aqueous salt solution.

15. The personal care product of claim 1 wherein the personal care product is selected from the group consisting of shaving products, antiperspirant products or deodorant products.

16. The personal care product of claim 1 wherein the propellant comprises a hydrocarbon oil.

17. The personal care product of claim 15 wherein the propellant has a vapor pressure of less than 125 psia at 70° F.

18. The personal care product of claim 1 wherein the product is dispensed from the container as a foam, and the foam density of the product remains substantially constant until at least 70% of the contents of the can have been exhausted.

19. The personal care product of claim 1 wherein the sorbant has a relatively higher affinity for the propellant than for the personal care formulation.

20. The personal care product of claim 1 wherein the propellant is substantially insoluble in the personal care formulation.

21. A personal care product comprising
a single compartment container, and, within the container, a personal care gel formulation and a propellant, wherein substantially all of the propellant is either sorbed onto a sorbant or present in its gaseous phase.

22. The personal care product of claim 21 wherein the sorbant has a relatively higher affinity for the propellant than for the personal care formulation.

23. The personal care product of claim 21 wherein the sorbant comprises a polymer.

24. The personal care product of claim 23 wherein the polymer is partially cross-linked.

25. The personal care product of claim 21 wherein the sorbant comprises a foam or a fibrous material.

26. The personal care product of claim 21 wherein the personal care gel formulation is selected from the group consisting of shaving gels, antiperspirant gels and deodorant gels.

27. A method of manufacturing a personal care product comprising:
(a) providing, within a container, a sorbant;
(b) delivering, to the container, a personal care formulation;
(c) sealing the container; and
(d) pressurizing the container with a propellant.

28. The method of claim 27 wherein the sorbant is delivered to the container with, before, or after the delivery of the personal care formulation.

29. The method of claim 27 wherein the sorbant is adhered to or coated on an inner surface of the container prior to delivery of the personal care formulation.

30. The method of claim 27 wherein the sorbant is adhered to or coated on a dip tube inserted into the container.
